(19) United States
(12) Patent Application Publication
(10) Pub. No.: US 2015/0037883 A1
(43) Pub. Date: Feb. 5, 2015

(54) METHOD FOR DERIVATION AND LONG-TERM ESTABLISHMENT OF GROUND STATE PLURIPOTENT EMBRYONIC STEM CELLS

(71) Applicants: Hossein Baharvand, TEHRAN (IR); Seyedeh Nafiseh Nafiseh Hasani, Tehran (IR); Mehdi Totonchi, Tehran (IR); Hamid Gourabi, Tehran (IR)

(72) Inventors: Hossein Baharvand, TEHRAN (IR); Seyedeh Nafiseh Nafiseh Hasani, Tehran (IR); Mehdi Totonchi, Tehran (IR); Hamid Gourabi, Tehran (IR)

(73) Assignee: ROYAN INSTITUTE, TEHRAN (IR)

(21) Appl. No.: 14/462,598

(22) Filed: Aug. 19, 2014

(51) Int. Cl. C12N 5/0735

(52) U.S. Cl. CPC C12N 5/0606
USPC 435/354

(57) ABSTRACT

The various embodiments herein provide a method for derivation and long term establishment of ground state pluripotent embryonic stem cells. Further the embodiments herein provides a method to inhibit the ERK and TGF β signalling pathways for long term maintenance of the embryonic stem cells. The R2i mouse embryonic stem (ES) cells are derived from 3.5 day blastocysts. The mouse ES cells are cultured in media containing R2i and 2i inhibitors of ERK and TGF β pathways. The ES cells are subjected to in vitro and in vivo differentiation. The ES cells are subjected to RT-PCR and qRT-PCR, flow cytometry and karyotyping. The result reveals that the R2i maintains the ground state of ES cells and self renewal. Also R2i increases embryonic cleavage and clonal propagation of ES. Further R2i asserts genomic integrity and pluripotency of ES.

### CROSS REFERENCE TO RELATED APPLICATION

The embodiments herein claims the priority of the U.S. Provisional Patent Application Ser. No. 61/771,928 on Mar. 3, 2013 with the title “A New Path to Efficient Establishment of Ground State Pluripotent Embryonic Stem Cells”, and the contents of which is incorporated by entirety as reference herein.

### BACKGROUND

1. Technical Field

The embodiments herein are generally related to a field of isolation and maintenance of the stem cells. The embodiments herein are particularly related to maintenance of the ground state of the mouse embryonic stem cells. The embodiments herein are more particularly related to a method of self renewal of the mouse embryonic stem cells by inhibition of ERK and TGF β signaling pathways. The embodiments herein also relate to a method of maintaining the pluripotency and karyotyping stability of the embryonic stem cells for at least 50 passages.

2. Description of the Related Art

Stem cells are undifferentiated biological cells that can be differentiated into specialized cells and divided to produce more stem cells. Basically the stem cells are found in multicellular organisms such as mammals. There are two broad categories of the stem cells called “embryonic stem cells” and “adult stem cells”. The embryonic stem cells are isolated from the inner cell mass of blastocyst and the adult stem cells are isolated from various tissues such as bone marrow.

In adult organisms, stem cells are progenitor cells which act as a repair system for the body, for replenishing an adult tissue. In the developing organisms, embryo stem cells can be differentiated into all the specialized cells such as ectoderm, endoderm and mesoderm. The embryonic stem cells also maintain the regeneration of organs, such as skin, blood regeneration or intestinal tissue repair.

The stem cells are cultured and maintained artificially in laboratory standard conditions. The main requirement for stem cell culture is a “culture media” or “culture medium”. The stem cells can be cultured either using a completely “natural medium” or an “artificial/synthetic medium” along with some natural products.

The natural media consist solely of naturally occurring biological fluids. The natural media are very useful and convenient for a wide range of animal cells cultures. The major disadvantage of natural media is its poor reproducibility due to lack of knowledge of the exact composition of these natural media.

The artificial or synthetic media are prepared by adding nutrients, vitamin, salt, oxygen and carbon dioxide gas phase, serum protein, carbohydrates, cofactors etc. Different artificial media have been devised to serve one or more of the following purpose: 1) immediate survival of the stem cells (a balanced salt solution, with specific pH and osmotic pressure), 2) prolonged survival of the stem cells (a balanced salt solution supplemented with various formulation of organic compounds and/or serum, 3) indefinite growth of the stem cells, and 4) specialized function.

Since the derivation of the first mouse embryonic stem cells lines in 1981, identifying the optimal culture condition have been as a big challenge for efficient generation and long term maintenance of these cells in undifferentiated state.

The conventional undefined culture conditions, including mouse embryonic fibroblasts (MEF) as feeder cells and serum, do not support embryonic stem cells generation from most of the mouse strains. Further the stem cells are exposed to a conflict between the expression of pluripotency factors and differentiation lineage marker genes. Hence these culture conditions are proper for 129 mouse strains and the other strains show recalcitrant attitude to embryonic stem cells derivation.

With the identification of the leukemia inhibitory factor (LIF) and the bone morphogenetic protein 4(BMP4), as the important factors in mouse embryonic fibroblasts and serum respectively; a defined feeder and serum free culture condition was established. But the generation of embryonic stem cells from refractory strains and acquiring the optimal culture condition for undifferentiated state of embryonic stem cells remained as a problem.

Later Smith and his coworkers attained a special strategy for efficient derivation and long term maintenance of mouse embryonic stem cells through using a small molecule inhibitors for fibroblast growth factor 4 (FGF4) signaling pathway and glycogen synthase kinase 3 (GSK3) pathway, PD0325901 and CHIR99021, respectively known as 2i. The 2i could bypass the strain type for embryonic stem cells generation throughout rodentia. Further the rate of heterogeneity with regards to morphology or expression of pluripotency factors is brought down by 2i. However, the long term stability of embryonic stem cells in 2i remains to be scrutinized, since GSK3 inhibitors such as CHIR have shown the induction of chromosomal instability.

Hence there is a need for an efficient and reproducible protocol for derivation and long-term maintenance of mouse embryonic stem cells by the use of small molecule inhibitors of extracellular signal regulated kinase (ERK) and transforming growth factor β (TGF β) signaling pathways, which does not cause instability in the stem cells. Also there is need for a method which does not induce chromosomal instability in the stem cells. Further there is need for a method which induces homogeneous expression of pluripotency factors in the embryonic stem cells. Also there is need for an efficient protocol which facilitates the generation of embryonic stem cells from different mouse strains with high efficiency and maintains embryonic stem cells in more homogeneously expression of pluripotency markers.

The above mentioned shortcomings, disadvantages and problems are addressed herein and which will be understood by reading and studying the following specification.

### OBJECTIVES OF THE EMBODIMENTS

The primary object of the embodiments herein is to provide an efficient and reproducible protocol/method for derivation and long term maintenance of mouse embryonic stem cells by the use of small molecule inhibitors of extracellular signal regulated kinase (ERK) and transforming growth factor β (TGF β) signaling pathways.

Another object of the embodiments herein is to provide a highly efficiency method to derive the embryonic stem cells from whole blastocysts and isolated inner cell masses (ICM) from different mouse strains in serum and serum feeder free condition.

Yet another object of the embodiments herein is to provide a new method for deriving embryonic stem cells from BALB/c, C57BL/6, DBA/2, F1 of C57BL/6 (Oct4−EGFP)×CD−1 mouse strains by R2i in the presence or absence of leukemia inhibitory factor (LIF) in the chemically defined medium.

Yet another object of the embodiments herein is to provide a new embryonic stem cell culture method in which the efficiency of the mouse embryonic stem cells derivation from single blastomeres of 2-cell, 4-cell and 8-cell embryos from different mouse strains in R2i supplemented media is 2 fold more than 2i supplemented media.

Yet another object of the embodiments herein is to provide a new embryonic stem cells culture method in which the mouse embryonic stem cells generated in culture media comprising R2i are homogenous in terms of the expression of pluripotency markers such as Nanong and Stella.

Yet another object of the embodiments herein is to provide a new embryonic stem cell culture method in which the embryonic stem cells grow in culture media comprising R2i have less differentiation leakage (expression of lineage differentiation marker genes such as Lefty 1, Lefty 2, Brachyury) in comparison with serum or 2i-contained medium.

Yet another object of the embodiments herein is to provide a new embryonic stem cell culture protocol to obtain the chromosomal stability which is much higher in the culture media consisting of R2i than media consisting of 2i media.

Yet another object of the embodiments herein is to provide a new embryonic stem cell culture protocol in which the transforming growth factor β (TGF β) receptor inhibition in combination with leukemia inhibitory factor (LIF) support's the ground state of the embryonic stem cells self renewal.

These and other objects and advantages of the embodiments herein will become readily apparent from the following detailed description taken in conjunction with the accompanying drawings.

### SUMMARY

The various embodiments herein provide a method for derivation and long-term establishment of ground state pluripotent embryonic stem cells. The embodiments herein provide a method of isolation and maintenance of the stem cells. The embodiments herein a method for the maintenance of the ground state of the mouse embryonic stem cells. The embodiments herein provide a method of self renewal of the mouse embryonic stem cells by inhibition of ERK and TGF β signaling pathways. The embodiments herein also provide a method of maintaining the pluripotency and karyotyping stability of the embryonic stem cells for at least 50 passages.

According to one embodiment herein, a method is provided for deriving and long term establishment of ground state pluripotent embryonic stem cells. The method comprises the steps of isolating mouse embryonic stem cells, and culturing the mouse embryonic stem cells in a culture media consisting of the small molecule inhibitors of signal regulated pathways, for a long term maintenance of the mouse embryonic stem cell.

According to one embodiment herein, the method for isolating and long term maintenance of mouse embryonic stem cell comprises the steps of maintaining a plurality of mouse strains on a 12 hour light/dark regimen. The mouse strains are selected from a group consisting of BALB/c, C57BL/6, DBA/2, and F1 generation of C57BL/6 (Oct−EGFP)×CD−1. Next the mouse embryos are recovered by flushing uteri or oviduct of a mouse for blastocysts or cleavage embryos at E3.5 stage. The blastocyst is obtained at E3.5 stage from the plurality of mouse strains. The mouse embryonic stem cells from 3.5 day blastocyst are derived in a media comprising R2i molecule. The zona-free E.3.5 day blastocyst is plated on a gelatine coated plate comprising a predefined medium with the R2i and a leukemia inhibitory factor (LIF), and the predefined medium is selected from a group consisting of KSOM medium, N2B27 medium and serum. The tropectoderm is removed by accomplishing immunological surgery for establishing the embryonic stem cell lines. Further a cell mass is disaggregated by a 0.05% trypsin 5-7 days after the blastocyst or an inner cell mass plating to obtain the disassociated cells. The disassociated cells are centrifuged to remove the serum and harvest the cells. The harvested cells are transferred in the bacterial dishes comprising N2B27 medium, the R2i molecule, and the LIF for cultivation as suspension.

According to one embodiment herein, the harvested cells are transferred on the gelatine coated plates comprising the N2B27 medium, the R2i molecule and the LIF for cultivation as adherent.

According to one embodiment herein, the small molecule inhibitor is R2i molecular combination and 2i molecular combination. The 2i molecular combination comprises a PD0325901 and a CHIR99021 molecule. Whereas the R2i molecular combination comprises a SB431542 and a PD0325901 molecule.

According to one embodiment herein, the small molecule inhibitor inhibits metabolic pathways, such as an extracellular signal regulated kinase (ERK) pathways and a transforming growth factor β (TGFβ) signalling pathway.

According to one embodiment herein, 100 ml of the N2B27 medium comprises 45 ml of Dulbeccos modified eagle medium-nutrient mixture F-12, 45 ml of neuro basal medium, 1 ml of nitrogen supplement (1 vol %), 2 ml of B27 supplement (1 vol %), 1 ml of L-glutamine (2 mM), 1 ml of non-essential amino acid (1 vol %), 1 ml of penicillin/streptomycin, B mercaptoethanol (0.1 mM), 5 mg/mL of Bovine serum albumin (BSA), 1000 μ/ml of leukemia inhibitory factor (LIF), and the R2i molecular combination. The R2i molecular combination comprises 1 μM PD 0325901 and 10 μM SB431542.

According to one embodiment herein, the derived embryonic stem cells are cultured separately on a mouse embryonic fibroblast (MEF) coated 96 well plate in a desired medium. The desired medium is selected from a group consisting of KSOM, mouse embryonic stem cells with serum medium and N2B27 medium supplemented with the 2i molecular combination or the R2i molecular combination.

According to one embodiment herein, the embryonic stem cells are routinely passaged in every 2-3 days. The mouse embryonic stem cells derived from the single blastomeres of 2 cell, 4 cell and 8 cell embryos from the plurality of mouse strains in the R2i molecular combination supplemented media is two times more than that of the 2i molecular combination supplemented media. Also the mouse embryonic stem cells generated in the culture media comprising the R2i molecular combination are homogeneous in terms of expression of pluripotency markers, and wherein the pluripotency markers comprise a Nanong and a Stella. The chromosomal stability of the embryonic stem cells in culture media comprising of the R2i molecular combination is higher than that in the media comprising of the 2i molecular combination.

According to one embodiment herein, an inhibition of transforming growth factor β (TGFβ) receptors in combination with the leukemia inhibitory factor (LIF) supports the ground state of the embryonic stem cells and a self renewal of the embryonic stem cells.

According to one embodiment herein, the gelatine coated plate comprises only the predefined medium with the R2i, and the predefined medium is selected from a group consisting of KSOM medium, N2B27 medium and serum.

The method for deriving and long-term establishment of ground state pluripotent embryonic stem cells comprises the small molecule inhibitors for inhibition of an extracellular signal regulated kinase (ERK) pathways and a transforming growth factor β (TGFβ) signalling pathway. Further the method involves the small molecule inhibitor i.e. R2i molecule combination and 2i molecule combination. The 2i molecule combination comprises of a PD0325901 and a CHIR99021. The R2i molecule combination consists of a SB431542 and a PD0325901.

The method for deriving and long-term establishment of ground state pluripotent embryonic stem cells has the following steps for the isolation and long term maintenance of mouse embryonic stem cell:maintaining the mouse strains on a 12 hour light/dark regimen; recovering the mouse embryo by flushing uteri or the oviduct for blastocysts or the early cleavage embryos; obtaining blastocyst at E3.5 from different mouse strains; deriving mouse embryonic stem cells from 3.5 day blastocyst in a media comprising R2i; plating the zona-free E.3.5 day blastocyst on a gelatine coated plate comprising a N2B27 defined medium with the R2i and with or without a leukaemia inhibitory factor (LIF); accomplishing immunology for the removal of tropectoderm and establishing embryonic stem cell lines; disaggregating the cell mass by a 0.05% trypsin 5-7 days after a blastocyst or an inner cell mass plating; centrifuging the disassociated cells to remove the serum and the harvested cells; transferring the harvested cells in bacterial dishes with the N2B27 medium+the R2i+the LIF for cultivation as suspension; or passing the harvested cells on gelatine coated plates with the N2B27 medium+the R2i+the LIF for cultivation as adherent.

Further the N2B27 media (100 ml) comprises: 45 ml Dulbeccos modified eagle medium-nutrient mixture F-12; 45 ml neuro basal medium; 1 ml nitrogen supplement (1%); 2 ml B27 supplement (1%); 1 ml L-glutamine (2 mM); 1 ml non-essential amino acid (1%); 1 ml penicillin/streptomycin; β mercaptoethanol (0.1 mM); 5 mg/mL Bovine serum albumin (BSA); 1000 μ/ml leukaemia inhibitory factor (LIF); and R2i molecule combination, wherein R2i molecule combination comprises of 1 μM PD 0325901 and 10 μM SB431542.

The derived embryonic stem cells are cultured separately on the mouse embryonic fibroblast (MEF) coated 96 well plate in desired medium; and the media is selected from KSOM or mouse embryonic stem cells (with serum) medium and/or N2B27 defined medium is supplemented with the 2i molecule combination or the R2i molecule combination. The embryonic stem cells are routinely passaged in every 2-3 days.

The mouse embryonic stem cells derivation from single blastomeres of 2 cell, 4 cell and 8 cell embryos from different mouse strains in the R2i molecule combination supplemented media is two times more than the 2i molecule combination supplemented media.

The mouse embryonic stem cells generated in the culture media comprising the R2i molecule combination are homogeneous in terms of the expression of pluripotency markers such as Nanong and Stella. The chromosomal stability is higher in culture media comprising of the R2i molecule combination than the media comprising of the 2i molecule combination. The transforming growth factor β (TGFβ) receptors inhibition in combination with the leukaemia inhibitory (LIF) supports the ground state of the embryonic stem cells self renewal.

According to one embodiment herein, an efficient and a reproducible protocol for the derivation and long term maintenance of the mouse embryonic stem cells is achieved by using small molecule inhibitors for an extracellular signal regulated kinase (ERK) and a transforming growth factor β (TGF β) signaling pathways. The small inhibitor molecules are a PD0325901 and a SB431542 respectively. The inhibitor molecule combination is named as a R2i and a 2i.

The 2i molecule combination consists of the small molecules such as a PD0325901 and a CHIR99021. The CHIR99021 is a potent inhibitor of glycogen synthase kinase 3 (GSK3). The 2i molecule combination causes the suppression of endogenous differentiation-inducing signaling. By introducing the 2i molecule combination, an efficient derivation of embryonic stem cells from different mouse strains has been possible. The 2i molecule combination causes GSK3 inhibition and leads to chromosomal abnormalities. To support ground state pluripotency in the embryonic stem cells, R2i molecule combination is used. It is found that when the TGF β signaling pathway is inhibited, the other differentiation is induced by signaling in mouse embryonic stem cells. This is achieved by a SB431542 coupled with inhibition of the fibroblast growth factor [FGF] such as PD0325901. The molecule combination of the SB431542 and the PD0325901 is called the R2i molecule combination or R2i. By using the R2i molecule combination, the ground state pluripotency is achieved.

The R2i molecule combination supports genome integrity of mouse embryonic stem cells from single blastomeres of early cleavage embryos nearly two times more efficient than that of the 2i.

According to one embodiment herein, the steps in the method for isolation and maintenance of mouse embryonic stem cells involve maintaining the mouse strains on a 12 hour light/dark regimen. The mouse embryos are recovered by flushing from the uteri or the oviduct for the blastocysts or early cleavage embryos respectively. The blastocyst at E3.5 is obtained from different mouse strains or from the breeding-cross of different mouse strains.

According to one embodiment herein, the next step is the derivation of the mouse embryonic stem cells from the 3.5 day blastocyst in a media comprising the R2i. The mouse embryonic stem cells are derived at blastocyst stage is done by plating the zona-free E3.5 embryos on gelatin coated plate (0.1% Sigma Aldrich) comprising the N2B27 defined medium with the R2i with or without (leukemia inhibitory factor) LIF. The immune-surgery is accomplished for the establishment of embryonic stem cell lines. Five to seven days after blastocysts or inner cell mass plating, the cell mass is disaggregated by a 0.05% trypsin. The trypsin is neutralized by mouse embryonic stem cell medium containing fetal bovine serum (FBS). The dissociated cells are centrifuged to remove the serum and harvested cells are transferred in the bacterial dishes with N2B27+R2i±LIF for cultivation as suspension, or alternatively the dissociated cells are passed on the gelatin coated plates contained N2B27+R2i±LIF for cultivation as adherent in that fresh defined medium are replaced for removing remnant serum after adhesion of cells.

According to one embodiment herein, the N2B27 media (100 ml) comprises of 45 ml Dulbecco's Modified Eagle Medium: Nutrient Mixture F-12 (DMEM/F12) (Invitrogen™), 45 ml neuro-basal medium (Invitrogen™), 1 ml nitrogen supplement (1%, Invitrogen™), 2 ml B27 supplement (1%, Invitrogen™), 1 ml L-glutamine (2 mM, Invitrogen™), 1 ml non-essential amino acids (1%, Invitrogen™), 1 ml penicillin/streptomycin (Invitrogen™), β-mercaptoethanol (0.1 mM, Sigma-Aldrich), 5 mg/mL bovine serum albumin (BSA) (Sigma-Aldrich®) and 1000 IU/ml leukemia inhibitory factor (LIF) (Millipore®). The small molecule i.e. R2i includes 1 μM of PD0325901 and 10 μM SB431542.

According to one embodiment herein, the derivation of the mouse embryonic stem cells from the single blastomers of the zona free 2, 4 and 8 cell stage mouse embryos are separated by gentle pipetting on fresh culture medium comprising the R2i. The single blastomeres are cultured separately on the mouse embryonic fibroblast (MEF) coated 96-well plates in desired medium. The media are KSOM, mouse embryonic stem cells (serum) medium and the N2B27 defined medium supplemented with the 2i or R2i.

According to one embodiment herein, the mouse embryonic stem cells, irrespective of the way of generating, are cultured without mouse embryonic fibroblast (MEF) on gelatin coated plates. The embryonic stem cells cultured in the presence of the R2i are routinely passaged in every 2-3 days.

According to one embodiment herein, the alkaline phosphatase activity and the immune-fluorescence staining is performed for the embryonic stem cells. The alkaline phosphatase activity is detected using a kit or the standard protocol. The immune-fluorescence for cultured cells is performed after fixation in 4% paraformaldehyde for 20 min, permeabilization with 0.2% Triton X-100 for 30 min and blocking in phosphate buffer saline supplemented with 10% goat serum for 1 hour. The embryonic stem cells are incubated with the primary antibodies overnight at 4° C. and are washed, incubated with secondary antibodies. The embryonic stem cells nuclei are counterstained with a 4′,6-diamidino-2-phenylindole (DAPI). The embryonic stem cells are visualized using the Olympus fluorescent microscope.

According to one embodiment herein, the embryonic stem cells are subjected to the in vitro and in vivo differentiation. The in vitro differentiation is performed as spontaneous by embryoid body (EB) formation or by induced differentiation. For the embryoid body formation, embryonic stem cells are dissociated by a trypsin. The stem cells are then cultured in the bacterial dishes consisting of a mouse embryonic stem cells medium without leukemia inhibitory factor (LIF). The embryoid bodies are plated onto the gelatinized plates for 7 days. The embryoid bodies are then assayed for the expression of pluripotency or lineage specifier genes. For the induced differentiation into neuronal lineages, 3 days embryoid bodies are transferred in medium containing 2 μM retinoic acid for 1 week, plated on gelatinized plates and assayed 3-4 days later. For the cardiomycocyte differentiation, the embryonic stem cells are trypsinized and cultured at density of 800 cells/20 μl hanging drops for 2 days in mouse embryonic stem cells medium without leukemia inhibitory factor (LIF), supplemented with 0.1 μM ascorbic acid. Subsequently the embryonic stem cells are transferred in to a bacterial dish for another 3 days and then rinsed onto gelatinized plates. The embryonic body formation is performed in the N2B27 medium for 3-4 days. For the teratoma formation, the embryonic stem cells are resuspended in Matri® gel and injected into syngenic mice and the growth is monitored. The tumor masses are stained with hematoxillin and eosin for all histological determinations.

According to one embodiment herein, the embryonic stem cells cultured in the media consisting R2i or 2i are subjected to flow cytometry. The trypsinized embryonic stem cells are fixed with ice cold methanol and blocked by 2% normal goat serum for 60 minutes washing and incubated with primary antibody. The embryonic stem cells are washed again and treated with a secondary antibody. After final washing a flow cytometric analysis is performed using FACS Calibur Flow Cytometer. The acquired data is analyzed using BD CellQuest™ Pro software.

According to one embodiment herein, for the karyotype analysis, the embryonic stem cells are treated with thymidine for 12 hours at 37° C., washed with fresh medium and 4 hours later treated with colcemid for 30 min. The embryonic stem cells are trypsinized swelled with potassium chloride (KCl) for 15 minutes. The embryonic stem cells are then fixed in the ice cold methanol and acetic acid mixture and dropped onto chilled slides. The chromosomes are visualized using standard G-band staining. The chromosome number of 50 metaphase spreads are screened by microscopy and counted on the micrographs.

According to one embodiment herein, the derivation and long term establishment of ground state pluripotent embryonic stem cells involves the following steps: 1) isolating a 3.5 day mouse blastocyct, 2) removing the zona pellucid from the isolated blastocyst, 3) removing the trophectoderm using immunosurgery, 4) culturing the whole blastocyst/inner cell mass (ICM) in gelatin (0.1%) coated dish and N2B27+PD032+SB43+leukemia inhibitory factor (LIF), for the 4-6 days, 5) pick up inner cell mass (ICM) outgrowth using mouth pipette, 6) washing the inner cell mass (ICM) outgrowth with phosphate saline (PBS) for 1 minutes, 7) trypsinizing the inner cellular mass (ICM) [0.05% trypsin-EDTA] for 2-3 minutes, 8) Culturing the cells in gelatin (0.1%) coated dish and N2B27+PD032+SB43+leukemia inhibitory factor (LIF), and 9) passaging the cells every 2-3 days.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a flow chart indicating a process of derivation and culture of mouse embryonic stem cells from 3.5 day blastocyst in a media comprising R2i, according to an embodiment herein.

FIG. 2 illustrates a bar chart indicating the derivation efficiency of continuous mouse embryonic stem cell lines under chemically defined medium supplemented with R2i, according to an embodiment herein.

FIG. 3A illustrates a bar chart indicating mRNA fold change of differentiated cells with respect to corresponding embryonic stem (ES) cells, according to an embodiment herein.

FIG. 3B illustrates a bar chart indicating is a bar chart illustrating indicating mRNA fold change of embryonic stem (ES) cells in 2i and R2i with respect to serum conditions, according to one embodiment herein.

FIG. 4A illustrates a bar chart indicating the results of an analysis of exclusion of the possible destructive effect of N2B27 basal medium during embryonic cleavage and clonally propagation of embryonic stem cells from single blastomeres, in development of embryonic cleavage according to one embodiment herein

FIG. 4B illustrates a bar chart indicating the embryonic stem cell generation from single blastomeres in R2i supplemented medium and the 2i supplemented medium in NMRI and BALB/c mouse cell lines, according to one embodiment herein.

FIG. 5 illustrates a bar chart indicating the role of R2i in genomic integrity after long term cultivation, according to the embodiments herein.

FIG. 6A illustrates a bar chart indicating the results of TGF β inhibition sustaining the pluripotency of mouse embryonic stem cells, according to an embodiment herein.

FIG. 6B illustrates a bar chart indicating the mRNA fold changes in stem cells during the knockdown of Smad2 by the use of siRNA in embryonic stem cells, according to one embodiment herein.

FIG. 6C illustrates a bar chart indicating the mRNA fold changes in stem cells during the knockdown of Smad3 by the use of siRNA in embryonic stem cells, according to one embodiment herein.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a method for derivation and long-term establishment of ground state pluripotent embryonic stem cells. The embodiments herein provide a method of isolation and maintenance of the stem cells. The embodiments herein provide a method for the maintenance of the ground state of the mouse embryonic stem cells. The embodiments herein provide a method of self renewal of the mouse embryonic stem cells by inhibition of the ERK and the TGF β signaling pathways. The embodiments herein also provide a method of maintaining the pluripotency and the karyotyping stability of the embryonic stem cells for at least 50 passages.

According to one embodiment herein, an efficient and reproducible protocol for derivation and long term maintenance of the mouse embryonic stem cells is achieved by using the small molecule inhibitors for an extracellular signal regulated kinase [ERK] and a transforming growth factor β [TGF β] signaling pathways. The small inhibitor molecules are a PD0325901 and a SB431542 respectively. The inhibitor molecule combination is named as a R2i and a 2i.

The 2i molecule combination consists of the small molecules such as a PD0325901 and a CHIR99021. The CHIR99021 is a potent inhibitor of glycogen synthase kinase 3 [GSK3]. The 2i molecule combination causes the suppression of endogenous differentiation-inducing signaling. By introducing the 2i molecule combination, an efficient derivation of embryonic stem cells from different mouse strains is possible. The 2i molecule combination causes the GSK3 pathway inhibition and leads to chromosomal abnormalities. To support a ground state pluripotency in the embryonic stem cells, the R2i molecule combination is used. It is found that when the TGF β signaling pathway is inhibited, the other differentiation is induced by signaling in mouse embryonic stem cells. This is achieved by the SB431542 coupled with inhibition of the fibroblast growth factor [FGF] i.e. PD0325901. The molecule combination of SB431542 and PD0325901 is called a R2i. By using the R2i molecule combination, the ground state pluripotency is achieved.

The R2i molecule combination supports the genome integrity of mouse embryonic stem cells from single blastomeres of early cleavage embryos nearly two fold more efficient then the 2i.

According to one embodiment herein, the steps in the method for isolation and maintenance of mouse embryonic stem cells involve maintaining the mouse strains on a 12 hour light/dark regimen. The mouse embryos are recovered by flushing from the uteri or the oviduct for obtaining blastocysts or early cleavage embryos respectively. The blastocyst at E3.5 is obtained from different mouse strains or from the breeding-cross of the different mouse strains.

According to one embodiment herein, the next step is derivation of the mouse embryonic stem cells from 3.5 day blastocyst in a culture medium comprising the R2i. The mouse embryonic stem cells are derived at the blastocyst stage is done by plating the zona-free E3.5 embryos on a gelatin coated plate (0.1% Sigma Aldrich) comprising the N2B27 defined medium with the R2i with or without the leukemia inhibitory factor (LIF). The immunosurgery is accomplished for establishment of embryonic stem cell lines. Five to seven days after the blastocysts or inner cell mass plating, the cell masses are disaggregated by a 0.05% trypsin. The trypsin is neutralized by mouse embryonic stem cell medium comprising the fetal bovine serum (FBS). The dissociated cells are centrifuged to remove the serum and harvested cells transferred in bacterial dishes with N2B27+R2i±LIF for cultivation as suspension, or alternatively the dissociated cells are passed on gelatin coated plates contained N2B27+R2i±LIF for cultivation as adherent in that fresh defined medium are replaced for removing remnant serum after adhesion of cells.

According to one embodiment herein, 100 ml of the N2B27 media comprises 45 ml of Dulbecco's Modified Eagle Medium: Nutrient Mixture F-12 (DMEM/F12) (Invitrogen™), 45 ml of Neurobasal® medium (Invitrogen™), 1 ml of nitrogen supplement (1%, Invitrogen™), 2 ml of B27 supplement (1%, Invitrogen™), 1 ml of L-glutamine (2 mM, Invitrogen™), 1 ml of non-essential amino acids (1%, Invitrogen™), 1 ml of penicillin/streptomycin (Invitrogen™), β-mercaptoethanol (0.1 mM, Sigma-Aldrich), 5 mg/mL of serum albumin (BSA) (Sigma-Aldrich) and 1000 IU/ml of leukemia inhibitory factor (LIF) (Millipore). The small molecule such as R2i includes 1 μM of PD0325901 and 10 μM of SB431542.

According to one embodiment herein, the function/role of each component in the culture medium is as follows. Dulbecco's Modified Eagle Medium comprising Nutrient Mixture F-12 (DMEM/F12) and Neurobasal® media are used for supporting the growth of mouse embryonic stem cells. Nitrogen supplement, chemical supplement, and serum free supplement are used with Neurobasal® media. B27 is an optimized serum substitute and contains a cocktail of antioxidants to reduce reactive oxygen damage. The L-glutamine which is an essential amino acid and nutrient in cell cultures is used for energy production and protein and nucleic acid synthesis. The Non-essential amino acids improve the growth and viability of propagating cells in culture while reducing the biosynthetic burden on these cells. Penicillin/Streptomycin is used as antibiotics against culture media contamination. Bovine Serum Albumin is used as an additive to cell culture media, especially serum free media. It provides a range of benefits including protection from oxidative damage and stabilization of other media components such as fatty acids. β-mercaptoethanol is used to reduce disulfide bonds and act as a biological antioxidant by scavenging hydroxyl radicals. Leukemia Inhibitory Factor (LIF) is used as the self renewal factor in mouse embryonic stem cell culture. The LIF activates the STAT3 transcription factor and P13 kinase enhances self renewal in mouse embryonic stem cells.

According to one embodiment herein, the derivation of R2i mouse embryonic stem cells from single blastomers of the zona free 2, 4 and 8 cell stage mouse embryos are separated by a gentle pipetting on the fresh culture medium. The single blastomeres are cultured separately on mouse embryonic fibroblast (MEF) coated 96-well plates in desired medium. The media are KSOM, mouse embryonic stem cells (serum) medium and the N2B27 defined medium supplemented with the 2i or R2i.

According to one embodiment herein, the mouse embryonic stem cells, irrespective of the way of generation, are cultured without mouse embryonic fibroblast (MEF) on the gelatin coated plates. The embryonic stem cells cultured in the presence of R2i are routinely passaged in every 2 days.

According to one embodiment herein, the alkaline phosphatase activity and the immune-fluorescence staining is performed for the embryonic stem cells. The alkaline phosphatase activity is detected using a kit. The immune-fluorescence for the cultured cells is performed after the fixation in a 4% paraformaldehyde for 20 min, permeabilization with a 0.2% Triton X-100 for 30 min and blocking in phosphate buffer saline supplemented with 10% goat serum for 1 hour. The embryonic stem cells are incubated with the primary antibodies overnight at 4° C. and then washed and incubated with secondary antibodies. The embryonic stem cells nuclei are counterstained with the 4′,6-diamidino-2-phenylindole (DAPI). The embryonic stem cells are visualized using the Olympus fluorescent microscope.

According to one embodiment herein, the embryonic stem cells are subjected to in vitro and in vivo differentiation. The in vitro differentiation is performed as spontaneous by embryoid body (EB) formation or by induced differentiation. For the embryoid body formation, embryonic stem cells are dissociated by a trypsin and cultured in bacterial dishes in mouse embryonic stem cells medium without leukemia inhibitory factor (LIF). The embryoid bodies are plated onto the gelatinized plates for 7 days. The embryoid bodies are then assayed for the expression of pluripotency or lineage specifier genes. For the induced differentiation into neuronal lineages, 3 days embryoid bodies are transferred in the medium containing 2 μM of retinoic acid for 1 week, plated on gelatinized plates and assayed 3-4 days later. For the cardiomycocyte differentiation, the embryonic stem cells are trypsinized and cultured at a density of 800 cells/20 μl of hanging drops for 2 days in mouse embryonic stem cells medium without leukemia inhibitory factor (LIF), and supplemented with 0.1 μM ascorbic acid. Subsequently the embryonic stem cells are transferred in a bacterial dish for another 3 days and then rinsed onto gelatinized plates. The embryonic body formation is performed in N2B27 medium for 3-4 days. For the tretoma formation, the embryonic stem cells are resuspended in Matri® gel and injected into syngenic mice and the growth is monitored. The tumor masses are stained with a hematoxillin and an eosin for all histological determinations.

According to one embodiment herein, the embryonic stem cells cultured in R2i or 2i media are subjected to a flow cytometry. The trypsinized embryonic stem cells are fixed with the ice cold methanol and blocked by 2% normal goat serum for 60 minutes, washed and incubated with primary antibody. The embryonic stem cells are washed again and treated with secondary antibody. After a final washing, a flow cytometric analysis is performed using the fluorescent calibur flow cytometry (FACS). The acquired data is analyzed using the BD CellQuest™ Pro software.

According to one embodiment herein, the embryonic stem cells are treated with thymidine for 12 hours at 37° C., washed with fresh medium and 4 hours later treated with colcemid for 30 min for the karyotype analysis. The embryonic stem cells are trypsinized and swelled with potassium chloride (KCl) for 15 minutes. The embryonic stem cells are then fixed in the ice cold methanol and acetic acid mixture and dropped onto the chilled slides. The chromosomes are visualized using standard G-band staining. The chromosome number of 50 metaphase spreads are screened by microscopy and counted on the micrographs.

According to one embodiment herein, the derivation and long term establishment of ground state pluripotent embryonic stem cells involves the following isolating a 3.5 day mouse blastocyct, removing the zona pellucida from the isolated blastocyst, removing the trophectoderm using immunosurgery, culturing the whole blastocyst/inner cell mass (ICM) in gelatin (0.1%) coated dish and N2B27+PD032+SB43+leukemia inhibitory factor (LIF), for the 4-6 days, picking up of inner cell mass (ICM) outgrowth using mouth pipette, washing the inner cell mass (ICM) outgrowth with phosphate saline (PBS) for 1 minutes, trypsinizing the inner cellular mass (ICM) [0.05% trypsin-EDTA] for 2-3 minutes, Culturing the cells in gelatin (0.1%) coated dish and N2B27+PD032+SB43+leukemia inhibitory factor (LIF), and passaging the cells in every 2-3 days.

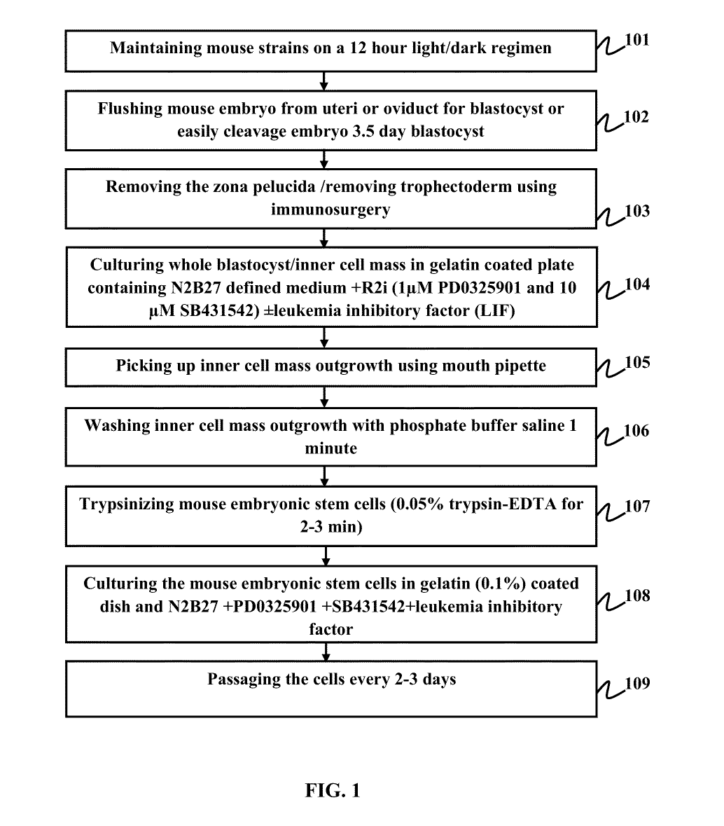

FIG. 1 illustrates a flow chart indicating the process of derivation and culture of mouse embryonic stem cells from 3.5 day blastocyst in a media comprising R2i, according to an embodiment herein. With respect to FIG. 1, the first step is maintaining mouse strains on a 12 hour light/dark regimen (101). The first step is followed by flushing the mouse embryo from uteri or oviduct for blastocyst or early cleavage embryo i.e. 3.5 day blastocyst (102). The next step is removing the zona pellucid/removing the trophectoderm using immunosurgery (103). The next step is culturing the whole blastocyst/inner cell mass in gelatin coated plate containing N2B27 defined medium+R2i (1 μM PD0325901 and 10 μM SB431542)±leukemia inhibitory factor (Lif) (104). The blastocyst are cultured for 4-6 days. Further, the inner cell mass outgrowth is picked up using mouth pipette (105). The inner cell mass outgrowth is subjected to washing with phosphate buffer saline for 1 minute (106). The next step is, trypsinising mouse embryonic stem cells (0.05% trypsin-EDTA for 2-3 minutes (107). The trypsinization is followed by culturing of the mouse embryonic stem cells in gelatin (0.1%) coated dish and N2B27+PD0325901+SB431542+Leukemia inhibitory factor (LIF) (108). The embryonic stem cells are passaged in every 2-3 days (109).

The passaging is important for mouse embryonic stem cells (mES) in every 2-3 days to maintain pluripotency. The steps involved in the passaging comprise aspiring the N2B27 supplemented medium including small molecules and leukemia inhibitory factor (LIF) from the wells containing expanded mES cells, washing the well with phosphate buffer saline and adding 100 μl trypsin/EDTA (0.05% w/v) solution to the well and incubating for 3-5 minutes in an incubator (37° C.), adding mouse embryonic stem cells in a medium containing fetal bovine serum (FBS) to inactive FBS trypsin activity, disassociating the cells into individual cells and small cell clumps, by pipetting up and down 10-20 times with a 1000 μl pipette tip, mixing the resulting single cell suspension with N2B27 supplemented medium should be changed daily, and splitting the mouse embryonic stem cells (mES) when they reach confluency. The mES cells are sub-cultured every 2-3 days.

### Experimental Details

### Materials and Methods

Animals and Embryos: The strains of mouse used in this study were maintained on a 12-hour light/dark regimen. The mouse strains were superovulated with standard protocol. Further all mouse embryos were recovered by flushing the uteri or the oviduct for collecting the blastocysts or early cleavage embryos, respectively. The Blastocysts at E3.5 were obtained from the cross of C57BL/6 (Oct4APE: GFP+ or OG2) and CD−1 (kind gift from Prof Scholer) or from the inbred mice C57BL/6, BALB/c and DBA/2 strains (Pasteus Institute of Iran). Early cleavage of embryos on 2, 4 and 8 cell stages were captured on 44 hour, 54 hour and 68 hour after hCG injection on out-bred NMRI or inbred BALB/c mice. The mouse embryonic fibroblasts (MEFs) that were used for embryonic stem (ES) cell derivation from mouse single blastomeres obtained from E13.5 fetuses from the NMRI strain.

Derivation of R2i Mouse Embryonic Stem (ES) Cells from 3.5 day Blastocysts: Derivation of mouse embryonic stem (ES) cells at blastocysts stage was done by plating the zona-free E3.5 embryos on gelatin coated plate (0.1%, Sigma-Aldrich) comprising N2B27 defined medium+R2i (Royan 2 inhibitors, include 1 μM PD0325901 (Stemgent®) and 10 μM SB431542 (Sigma-Aldrich®) with or without Leukemia inhibitory factor (LIF) (ESGRO, Millipore®). Alternatively, immunosurgery was also accomplished for establishment of ES cell lines. Five to seven days after blastocysts or inner cell mass (ICM) plating, the cell masses were disaggregated by 0.05% trypsin (Invitrogen®). The trypsin was neutralized by mouse ES cell medium (or “serum” medium) containing fetal bovine serum (FBS). The dissociated cell solution was centrifuged to remove the serum and the harvested cells were transferred in bacterial dishes with N2B27+R2i±LIF for cultivation as suspension, or alternatively enough amount of dissociated cells were passed on gelatin on gelatin coated plates comprising N2B27+R2i±LIF, for cultivation of the harvested cells as adherent layer. The fresh defined medium is replaced for removing the remnant serum after adhesion of the cells (usually 2-3 hours after plating).

The N2B27 medium comprises DMEM/F 12 (Invitrogen®) in 1:1 ratio, 1% N2 supplement (Invitrogen®), 1% B27 supplement (Invitrogen®), 2 mM of L-glutamine (Invitrogen®), 1% non-essential amino acids (Invitrogen®), 100 U/ml of penicillin and 100 mg/ml of streptomycin (Invitrogen), 0.1 mM of β-mercaptoethanol (Sigma-Aldrich®) and 5 mg/mL of bovine serum albumin (BSA) (Sigma-Aldrich®).

The Mouse ES (serum) medium comprises knockout Dulbecco's modified Eagle's medium (Invitrogen®), 15% fetal bovine serum (FBS, HyClone), 2 mM of L-glutamine, 1% non-essential amino acids, 100 U/ml of penicillin and 100 mg/ml of streptomycin, 0.1 mM of β-mercaptoethanol and 100 U/ml of mouse LIF.

Derivation of R2i Mouse Embryonic Stem (ES) Cells from Single Blastomeres: Single blastomeres of the zona-free 2, 4 and 8 cell mouse embryos were separated by gentle pipetting on fresh culture medium. The single blastomeres were cultured separately on mouse embryonic fibroblast (MEF) coated 96-well plates in desired medium. The medium are chosen from KSOM, mouse ES cell (serum) medium and N2B27 defined supplemented with 2i or R2i.

Culture of Mouse Embryonic Stem (ES) Cells: Mouse ES cells, irrespective of the way of generation, were cultured without mouse embryonic fibroblast (MEF), on gelatin coated plates. The R2i medium cultured cells are routinely passaged for every 2 days as the route mentioned earlier. Usually 1:3 ratio of cells is passaged in every culture batch. For transferring the R2i supplemented medium cells in 2i supplemented medium or serum, the N2B27+2i or mouse ES (serum) medium, respectively were used with or without LIF. The 2i composition includes 1 μM PD0325901 and 3 μM CHIR99021 (Stemgent).

Alkaline Phosphatase Activity and Immunofluorescence Staining: Alkaline phosphatase activity was detected using a kit (Sigma-Aldrich, 86R). The immunofluorescence for cultured cells was performed after fixation in 4% paraformaldehyde for 20 min, permeabilization with 0.2% Triton X-100 for 30 min and blocking in PBS supplemented with 10% goat serum for 1 h. The cells were incubated with the primary antibodies overnight at 4° C., washed, incubated with secondary antibodies. The nuclei were counterstained with DAPI (Sigma-Aldrich, D84170, 1 μg/ml). The cells were visualized using the Olympus fluorescent microscope (Olympus, Japan).

In Vitro and In Vivo Differentiation of Embryonic Stem (ES) Cells: In vitro differentiation was performed as spontaneously by embryoid body (EB) formation or by induced differentiation. For the EB formation, ES cells were dissociated by trypsin and cultured in bacterial dishes in mouse embryonic stem (ES) medium without leukemia inhibitory factor (LIF). Usually in 7 days, the EB's were plated onto gelatinized plates and 7 days later they were assayed for the expression of pluripotency or lineage specifier genes. For induction of differentiation into neuronal lineages, the 3 days EB's were transferred in a medium comprising 2 μM retinoic acid for 1 week. Further the EB's were plated on gelatinized plates and assayed 3-4 days later. For cardiomycocyte differentiation, the embryonic stem (ES) cells were trypsinized and cultured at a density of 800 cells/20 μl hanging drops for 2 days in mouse ES (serum) medium without LIF, and supplemented with 0.1 μM ascorbic acid. Subsequently the ES were transferred in bacterial dish for another 3 days and then rinsed onto gelatinized plates. Beating cells were observed usually 1-5 days after plating. For the endoderm lineage differentiation, the EB formation was performed in the N2B27 medium for 3-4 days and after that 50 nM Activin A is added for 5 days followed by plating onto gelatinized cell culture dishes and assayed 72 hours later. For teratoma formation, 3−5*10⁶ ES cells were resuspended in Matrigel® and injected subcutaneously into syngenic mice and teratoma growth was monitored. Paraffin sections of tumor masses were stained with Hematoxilin and Eosin (H&E) for all histological determinations. To generate the chimeric mice, dissociated ES cells were injected into the E 3.5 blastocysts. The chimeric mice were generated by standard protocol. Chimerism was determined by coat color in mice. To test for germ-line transmission, the chimeras were mated to the males or females from the injected strain of ES cells.

Reverse Transcription (RT) and Quantitative-Real Time (q-RT) PCR: For RT and qRT-PCR the standard protocol/procedure was followed.

Flow Cytometry: The trypsinized embryonic stem cells (ES) cells were fixed with ice cold methanol, blocked by 2% normal goat serum for 60 min, washed, and incubated with primary antibody (Oct4, Nanog and Stella) for 1 hour at 37° C. The ES cells were washed again and treated with secondary antibody for 30 min at 37° C. After final washing, flow cytometric analysis was performed using a FACS Calibur Flow Cytometer (BD Biosciences). As a negative control, the cells were stained with the appropriate isotype-matched control. The acquired data was analyzed using BD CellQuest™ Pro software.

Karyptype: For karyotype analysis, embryonic stem (ES) cells were treated with thymidine (0.66 mM, Sigma-Aldrich) for 12 hour at 37° C. The ES cells were washed with fresh medium and 4 hours later treated with colcemid (0.15 mg/ml, Invitrogen) for 30 min. The ES cells were trypsinized, swelled with KCl (75 mM) for 15 min, fixed in ice-cold methanol and acetic acid (3:1) and dropped onto chilled slides. The chromosomes were visualized using standard G-band staining. The chromosome number of 50 metaphase spreads were screened by microscopy and counted on the micrographs.

### Results and Discussion

Efficient Derivation of Continuous Mouse Embryonic Stem Cell Lines with R2i: To investigate the effect of R2i on generation of ES cells and maintenance of pluripotency in chemically defined medium, the first step is culturing the whole zona-free 3.5 day F1 blastocysts of C57BL/6 (Oct4ΔPE: GFP⁺ or OG2)×CD−1, on gelatin-coated 96-wells plate in N2B27 medium supplemented with R2i and tracing the procedure of line derivation in comparison with serum+LIF, as conventional culture medium. In the presence of R2i, inner cell mass (ICM) cells propagated while maintaining Oct4 expression after 6 days with few trophectodermal cells. In contrast, in the presence of serum, regardless of large ICM outgrowth, the GFP expression was decreased in the course of time and disappointed at 6^{th} day. In this condition, numerous trophectodermal and differentiated cells have surrounded the outgrowth. After five to seven days, GFP⁺ ICM outgrowths were dissociated enzymatically into single cells and replaced in the gelatin-coated plates contained N2B27 medium supplemented with R2i. Six days later, the undifferentiated colonies appeared were passaged continuously with the expression of Oct4-GFP. The procedure was extended to ES cell derivation from some other mouse strains which are refractory to ES generation under conventional conditions. The R2i also supported the efficient derivation of ES cells from DBA/2 and BALB/c (50-60% efficiency). To enhance the cloning efficiency, the leukemia inhibitory factor (LIF) was added to R2i during ES cell derivation procedure, as an important factor for clonogenicity. The R2i+LIF in the medium also support the highest efficiency for the derivation from different mouse strains including F1 of OG2×CD−1, C57BL/6, DBA2 and BALB/c.

All derived lines propagated repeatedly in the medium supplemented with R2i (“R2i” cells) as colonies of packed cells with high nucleus to cytoplasm ratio and obvious nucleoli characteristics of stable ES cell lines. The R2i cells (Embryonic stem cells grown/cultured in presence of R2i) were maintained for over 50 passages (±LIF) with no perceptible changes in growth rate or indications of senescence. The embryonic stem (ES) cells were also frequently cryopreserved and recovered with high efficiency by conventional techniques with DMSO as cryoprotectant. To interrogate the pluripotency identity of R2i cells, the expression of stem cell markers were examined in at least two randomly selected lines per each strain. All of the assessed ES cell lines demonstrated positive staining for alkaline phosphatase (ALP) and expressed Oct4, Nanog, and SSEA-1 expression. The development potential of selected R2i cells was also evaluated by in vitro and in vivo differentiation assays. RT-PCR analysis showed the ability of formed EBs to generate the derivatives of three embryonic germ layers. In addition, lineage specific differentiation protocols exhibited the capability of R2i cells to cells to form Map2⁺ and Tuj1⁺ neuronal lineage, GATA4⁺ mesendoderm, Mhc⁺ cardiomycocytes, and Foxa2⁺ and αFP⁺ endoderm. The differenatiation capacity of R2i cells was further assessed using chimera formation in colored C57BL/6 and DBA2 derived ES cells. All of the selected ES cell lines contributed to the chimera including the germline.

To assess whether other embryonic stem (ES) cells that have been derived or cultivated in serum+LIF (“serum” cells) are adaptable to R2i, the ES cells were transferred in R2i. Several ES cell lines tested by this new condition showed morphological adaptation to R2i after two to three passages of lines. The potential of chimera and germline transmission were also preserved after long-term passages in R2i, as Bruce4 ES cells showed it after 10 passaging in R2i with initial passage 18. Consequently, the obtained data showed that R2i prompt the generation of ES cells with highest efficiency from different strains and maintain self-renewal and pluripotency along several passaging that in this case LIF could be used optionally for restoring normal population doubling.

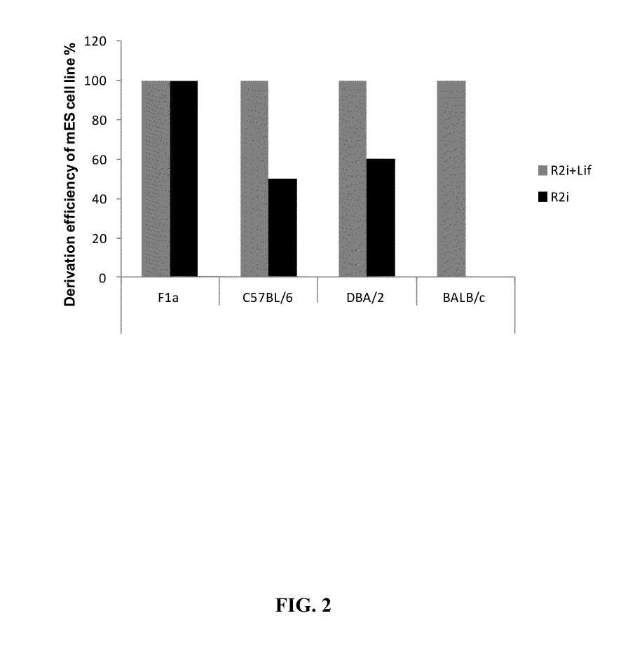

FIG. 2 illustrates a bar chart indicating the derivation efficiency of continuous mouse embryonic stem cell lines with R2i, according to an embodiment herein. With respect to FIG. 2, the derivation efficiency of mouse embryonic cell lines under chemically defined medium supplemented with R2i is higher when the media is supplemented with leukemia inhibitory factor (LIF) i.e. R2i+Lif. The derivation efficiency is compared with the chemically defined media supplemented with only R2i. FIG. 2 illustrates the mouse embryonic cell lines F1a, C57BL/6, DBA/2 and BALB/c cultured in chemically defined media with R2i+Lif and chemically defined media with only R2i. The bar chart (FIG. 2) illustrates that the derivation efficiency of the mouse embryonic stem cell (mES) line is 100%, when the mES cell lines are cultured in chemically defined media with R2i+LIF. The FIG. 2 further illustrates that when the chemically defined media is supplemented with only R2i, then the derivation efficiency is 100% only in the case of F1a mES cell line. The other cell lines i.e. C57BL and DBA/2 show 50% and 60% derivation efficiency respectively.

R2i maintains the ground state of Embryonic Stem Cell (ES) self-renewal: The improvement of culture condition during generation and maintenance of pluripotent stem cell lines has revealed the salient extrinsic control of pluripotency in recent years. With the introduction of 2i in the medium, the ability of ES cell generation bestows throughout recalcitrant rodent strains. ES cells show more homogeny in 2i than serum, so the subpopulations and cell-to-cell nonconformity in respect of Nanog and Rex1 expression get declined in 2i. As it was seen earlier, the R2i could also bypass strain type during ES cells derivation. For evaluation the homogeny of ES cells in R2i, two R2i cell lines were cultivated i.e. Royan B20 and Royan D4 in serum+LIF, 2i±LIF and R2i±LIF for 7 passages and examined the protein profile of Nanog and Stella by immunostaining and flow cytometry. The data revealed that although ES cells in different culture conditions showed the high expression of Oct4, but they are different in expression of Nanog and Stella as significantly are more uniform in 2i and R2i.

Recently it is shown that the transcriptional profile of embryonic stem (ES) cells do not exhibit “fixed” state as it could be “interconvertible” after transfer of 2i cell lines in serum and vice versa. The transcriptional state of R2i cell, Royan B20, that are cultivated simultaneously for 7 passages in serum, 2i and R2i supplemented medium (all plus LIF) are assessed for pluripotency and early lineage differentiation of key genes in each condition by qRT-PCR. High pluripotency-affiliated gene expression and low to absence of the most lineage-associated gene profile showed that R2i support the stable “ground state” in ES cells. The R2i diminished significantly the expression of various developmentally regulated genes, including Blimp1, Pax6, Fgf5, Brachyury, Lefty1, Lefty2, Sox7, Foxa2 and αFP. Although the unstable expression of pluripotency and lineage specifier genes is taken as the intrinsic ability of ES cells to beget differentiation into specified lineages, but these conditions usually reconcile with serum that could not be considered as optimum culture conditions. Although, it is shown that the R2i medium cultured cells have the ability to differentiate by direct differentiation or chimera and germline formation. The differentiation potential was assessed again of the R2i cultured stem cells by EB formation, and the results were compared when the stem cells were cultured at least 7 passages in serum and 2i supplemented medium by qRT-PCR. The data revealed that despite the lowest level of the most of lineage—affiliated genes of embryonic stem cells in R2i media, differentiation events presage properly after its removal. When taken together, protein and transcription profiles of archetype stem cell markers and lineage specifiers confirmed that R2i media cultured stem cells exhibited bonafide ground state of pluripotency.

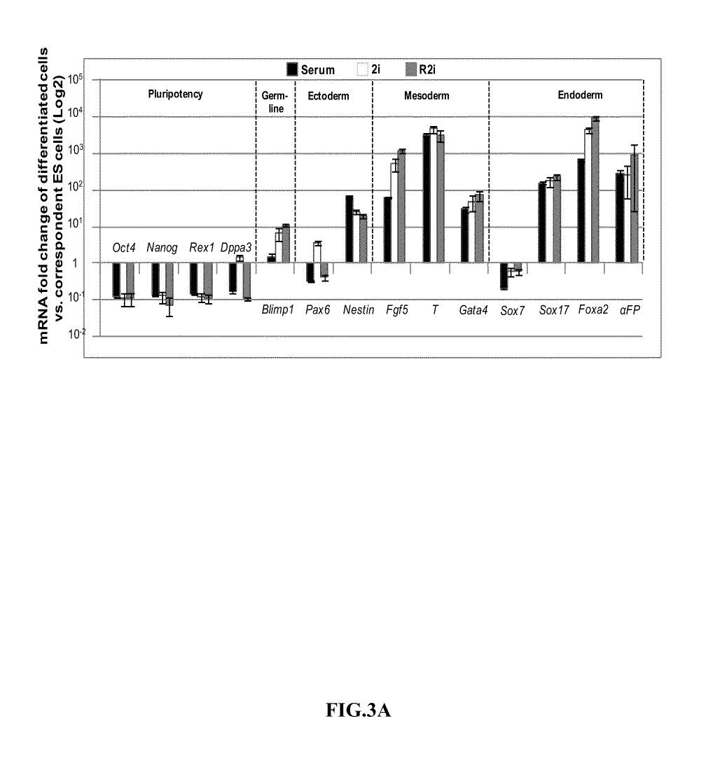

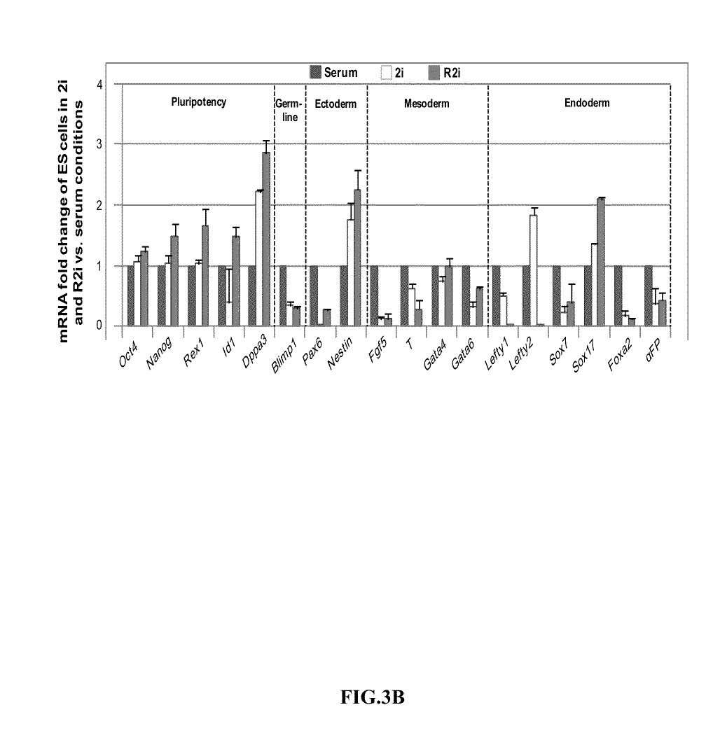

FIG. 3A and FIG. 3B illustrate a bar chart indicating the role of R2i in maintaining the ground state of embryonic stem cell self renewal, according to the embodiments herein. FIG. 3A illustrates a bar chart illustrating embryoid body (EB) differentiation potential (7 days EB plating) of Royan B20 that has been passaged seven times concurrently in serum, 2i and R2i [all treatments with leukemia inhibitory factor (LIF)] before EB formation, according to one embodiment herein. The expression levels of different genes associated with pluripotency and various germ layers (ectoderm, endoderm and mesoderm) are determined by qRT-PCR. Undifferentiated states of the stem cells in each condition are considered as the controls. The relative expression levels are normalized to the housekeeping gene GapDh. FIG. 3A illustrates that the expression of genes is higher in the case of embryonic stem cells cultured in chemically defined medium supplemented with R2i. The expression of the genes is less in the case of embryonic stem cells cultured in chemically defined medium supplemented with 2i. The FIG. 3A further illustrates that the expression of the genes is lowest in case of case of embryonic stem cells cultured in chemically defined medium supplemented with serum.

FIG. 3B illustrates a bar chart indicating the protein and transcriptional profiles of the archetype stem cells markers and lineage specifiers, according to one embodiment herein. The FIG. 3B illustrates the qRT-PCR of the genes associated with pluripotency and the various germlayers. The embryonic stem (ES) cells such as the Royan B20 ES cell lines are cultured for seven passages in chemically defined media with serum, 2i and R2i [all treatments with leukemia inhibitory factors (LIF)]. The ES cells cultured in media supplemented with serum are considered as control. Further FIG. 3B illustrates that the expression of the various genes associated with pluripotency and the various germ layers is higher in the ES cells cultured with R2i+LIF, than ES cells cultured with 2i+LIF.

R2i increases the development of embryonic cleavage and clonally propagation of Embryonic Stem cells from single blastomeres: The prevailing conception about the foundation of pluripotent stem cells is the “capturing” of “naive ground state” from the cells of preimplantation epiblast. However the potential of early embryonic blastomeres in developmental biology and the ability of them to pluripotent cell generation have not been fully annotated. To investigate the possibility that R2i also attains naive pluripotency from early cleavage stage mouse embryos, the embryonic stem (ES) cells derivation from single blastomeres at different developmental stages was analyzed. At first, to exclude the probability that N2B27 basal medium induce destructive effect on the embryonic cleavage, the embryo development of NMRI mouse strain from 2 cell stage towards intact hatched blastocyst in the droplets of different medium was analyzed. The medium assessed included KSOM, as conventional cleavage medium, serum+LIF and N2B27 basal medium alone or supplemented with 2i or R2i. It is found that N2B27 medium is equal to KSOM in progressing early embryo development. When R2i is added to N2B27, most of the early mouse embryos gain the potential to thrive from 2 cell stage to intact hatched blastocysts significantly more than other medium. Thereafter, single blastomeres from 2, 4 and 8 cell stage embryos of two strains, NMRI and BALB/c, were evaluated for ES cell generation in the presence of serum, 2i and R2i (each supplemented with LIF). Derivation of the embryonic stem cells in the level of single blastomeres was performed on individual mouse embryonic fibroblast (MEF)-coated 96 well plates, pursuant to the observation that the division ability of single blastomeres lost usually on gelatin. Table 1 below illustrates the derivation of embryonic stem cells from single blastomeres on gelatin coated plates:

TABLE 1
Table. Derivation of ES cells from single blastomeres on gelatin-coated
plates

| | |
|---|---|
| | Developed ES |
| Cleavage stage | cell lines (%) |

| | | | | |
|---|---|---|---|---|
| Replicate | Strain of mice | # of embryo | 2i | R2i |

2-cell

| | | | | |
|---|---|---|---|---|
| 1 | SW | 13 | 0 | 0 |
| 2 | NMRI | 15 | 0 | 0 |
| 3 | NMRI | 24 | 0 | 0 |

4-cell

| | | | | |
|---|---|---|---|---|
| 1 | NMRI | 12 | 0 | 0 |
| 2 | NMRI | 8 | 0 | 0 |

8-cell

| | | | | |
|---|---|---|---|---|
| 1 | NMRI | 1 | 0 | 0 |
| 2 | NMRI | 25 | 0 | 0 |
| 3 | NMRI | 16 | 0 | 0 |

In this situation, none of the single cells could attain pluripotency in serum contained medium from each cleavage stage. However, when N2B27 medium was supplemented with 2i or R2i, the acceptable growth of cells was observed and that usually lead to embryonic stem (ES) cell generation. Table 2 given below illustrates the derivation efficiency of embryonic stem cell lines from single blastomeres of 2-cell embryos on mouse embryonic fibroblast (MEF):

TABLE 2
Table. Derivation efficiency of ES cell lines from single blastomeres of 2-cell
embryos on MEF* NMRI Strain

| 2i | | | R2i | | |
|---|---|---|---|---|---|
| | # of dissociated | # of established | | # of dissociated | # of established lines/ |
| # of embryo | blastomeres | lines/embryo (%) | # of embryo | blastomeres | embryo (%) |
| 1 | 2 | 0 (0) | 1 | 2 | 0 (0) |
| 2 | 2 | 2 (100) | 2 | 2 | 2 (100) |
| 3 | 2 | 0 (0) | 3 | 2 | 2 (100) |
| 4 | 2 | 0 (0) | 4 | 2 | 2 (100) |
| 5 | 2 | 0 (0) | 5 | 2 | 0 (0) |
| 6 | 2 | 2 (100) | 6 | 2 | 2 (100) |
| 7 | 2 | 0 (0) | 7 | 2 | 1 (50) |
| 8 | 2 | 1 (50) | 8 | 2 | 2 (100) |
| 9 | 2 | 1 (50) | 9 | 2 | 2 (100) |
| 10 | 2 | 1 (50) | 10 | 2 | 0 (0) |
| 11 | 2 | 0 (0) | 11 | 2 | 2 (100) |
| 12 | 2 | 0 (0) | 12 | 2 | 0 (0) |
| 13 | 2 | 0 (0) | 13 | 2 | 2 (100) |
| 14 | 2 | 0 (0) | 14 | 2 | 1 (50) |
| 15 | 2 | 1 (50) | 15 | 2 | 0 (0) |
| 16 | 2 | 0 (0) | 16 | 2 | 0 (0) |
| 17 | 2 | 0 (0) | 17 | 2 | 2 (100) |
| Total | 34 | 8 | Total | 34 | 20 |

Total efficiency of established ES cell lines (%)

| | From dissociated | | From dissociated |
|---|---|---|---|
| From total embryos | blastomeres | From total embryos | blastomeres |
| 35 | 24 | 65 | 59 |

Table 3 illustrates the derivation efficiency of embryonic stem cell lines from single blastomeres of 4-cell embryos on mouse embryonic fibroblast:

TABLE 3
Table. Derivation efficiency of ES cell lines from single blastomeres
of 4-cell embryos on MEF*
NMRI Strain

| 2i | | | R2i | | |
|---|---|---|---|---|---|
| | # of | # of established | | # of | |
| | dissociated | lines/embryo | | dissociated | # of established |
| # of embryo | blastomeres | (%) | # of embryo | blastomeres | lines/embryo (%) |
| 1 | 3 | 1 (33.33) | 1 | 3 | 2 (66.67) |
| 2 | 3 | 2 (66.67) | 2 | 3 | 1 (33.33) |
| 3 | 3 | 0 (0) | 3 | 3 | 2 (66.67) |
| 4 | 3 | 0 (0) | 4 | 3 | 2 (66.67) |
| 5 | 3 | 1 (33.33) | 5 | 3 | 0 (0) |
| Total | 15 | 4 | Total | 15 | 7 |

Total efficiency of established ES cell lines (%)

| | From dissociated | | From dissociated |
|---|---|---|---|
| From total embryos | blastomeres | From total embryos | blastomeres |
| 60 | 27 | 80 | 47 |

BALB/c Strain

| 2i | | | R2i | | |
|---|---|---|---|---|---|
| | # of | # of established | | # of | |
| | dissociated | lines/embryo | | dissociated | # of established |
| # of embryo | blastomeres | (%) | # of embryo | blastomeres | lines/embryo (%) |
| 1 | 3 | 2 (66.67) | 1 | 4 | 2 (50) |
| 2 | 3 | 0 (0) | 2 | 3 | 1 (33.33) |
| 3 | 3 | 1 (33.33) | 3 | 3 | 3 (100) |
| 4 | 4 | 2 (50) | 4 | 3 | 1 (33.33) |
| 5 | 3 | 2 (66.67) | 5 | 4 | 2 (50) |
| 6 | 3 | 0 (0) | 6 | 4 | 3 (75) |
| 7 | 4 | 0 (0) | 7 | 3 | 3 (100) |
| Total | 23 | 7 | Total | 24 | 15 |

Total efficiency of established ES cell lines (%)

| | From dissociated | | From dissociated |
|---|---|---|---|
| From total embryos | blastomeres | From total embryos | blastomeres |
| 57 | 30.5 | 100 | 62.5 |

*The procedure of ES cell generation was also done in serum condition but it didn't lead to line derivation.

Table 4 below illustrates the derivation efficiency of embryonic stem cell lines from single blastomeres of 8-cell embryos on mouse embryonic fibroblast (MEF):

TABLE 4
Table. Derivation efficiency of ES cell lines from single blastomeres of
8-cell embryos on MEF*
NMRI Strain

| 2i | | | R2i | | |
|---|---|---|---|---|---|
| | # of | # of established | | # of | |
| | dissociated | lines/embryo | | dissociated | # of established |
| # of embryo | blastomeres | (%) | # of embryo | blastomeres | lines/embryo (%) |
| 1 | 8 | 0 (0) | 1 | 8 | 3 (37.5) |
| 2 | 8 | 2 (25) | 2 | 8 | 5 (62.5) |
| 3 | 8 | 4 (50) | 3 | 8 | 4 (50) |
| Total | 24 | 6 | Total | 24 | 12 |

Total efficiency of established ES cell lines (%)

| | From dissociated | | From dissociated |
|---|---|---|---|
| From total embryos | blastomeres | From total embryos | blastomeres |
| 67 | 25 | 100 | 50 |

BALB/c Strain

| 2i | | | R2i | | |
|---|---|---|---|---|---|
| | # of | # of established | | # of | |
| | dissociated | lines/embryo | | dissociated | # of established |
| # of embryo | blastomeres | (%) | # of embryo | blastomeres | lines/embryo (%) |
| 1 | 8 | 2 (25) | 1 | 8 | 3 (37.5) |
| 2 | 8 | 0 (0) | 2 | 8 | 2 (25) |
| 3 | 8 | 4 (50) | 3 | 8 | 3 (37.5) |
| 4 | 8 | 0 (0) | 4 | 8 | 5 (62.5) |
| 5 | 8 | 4 (50) | 5 | 8 | 4 (50) |
| 6 | 8 | 1 (12.5) | 6 | 8 | 5 (62.5) |
| Total | 48 | 11 | Total | 48 | 22 |

Total efficiency of established ES cell lines (%)

| | From dissociated | | From dissociated |
|---|---|---|---|
| From total embryos | blastomeres | From total embryos | blastomeres |
| 67 | 23 | 100 | 46 |

*The procedure of ES cell generation was also done in serum condition but it didn't lead to line derivation.

Interestingly, efficiency of ES cell generation in R2i supplemented medium showed nearly 2 fold increase relative to 2i. The selected R2i lines from each embryonic cleavage stages demonstrated the expression of archetype ES cell markers and the capability of chimera formation. The present findings revealed that naive ES cells also develop and expand in R2i from single blastomeres of early mouse embryo with efficiency more than well-known “2i” condition.

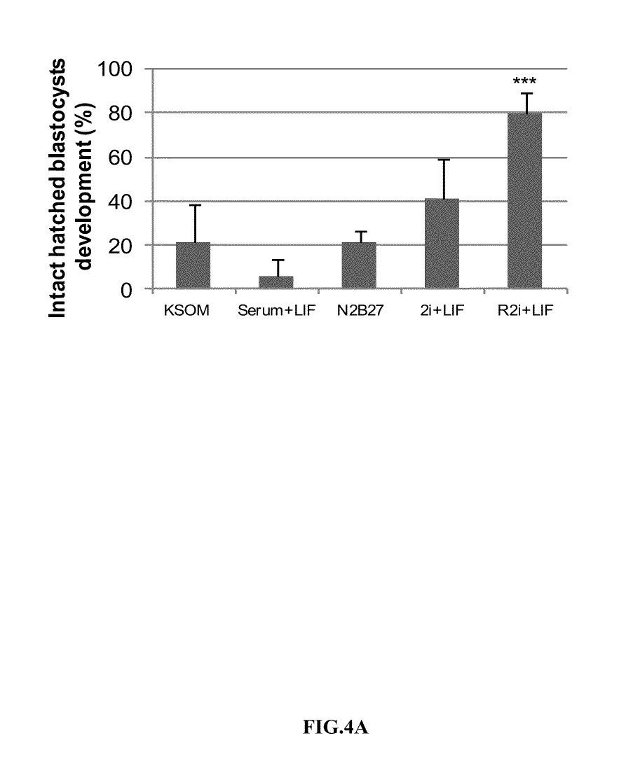

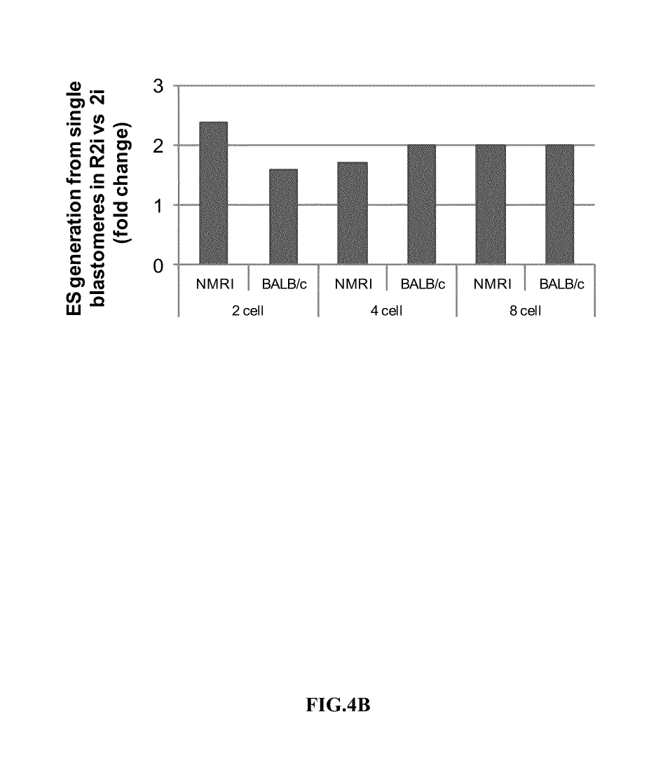

FIG. 4A-4B illustrate a bar chart indicating the role of R2i in development of embryonic cleavage and clonally propagation of embryonic stem cells from single blastomeres, according to the embodiments herein.

FIG. 4A illustrates a bar chart indicating the analysis of exclusion of the possible destructive effect of N2B27 basal medium during embryonic cleavage, according to one embodiment herein. The development of 2 cell stage embryos towards intact hatched blastocysts in droplets of different medium that includes KSOM (conventional cleavage medium), serum+LIF, N2B27 basal medium, 2i+LIF and R2i+LIF. The FIG. 4A illustrates that the N2B27 medium is similar to KSOM medium, both the medium allow progressive early embryo development.

FIG. 4B illustrates a bar chart indicating the embryonic stem cell generation from single blastomeres in R2i supplemented medium and the 2i supplemented medium in NMRI and BALB/c mouse cell lines, according to one embodiment herein. FIG. 4B illustrates that the single blastomeres from 2, 4 and 8 cell embryo of NMRI and BALB/c strains are capable of generating embryonic stem cells in serum, medium+2i and medium+R2i in the presence of leukemia inhibitory factor (LIF). FIG. 4B further illustrates that the grown blastomeres generated embryonic stem cells in medium+2i+LIF and R2i+LIF are nearly two folds more than the embryonic stem cell lines generated in the presence of R2i than in presence of 2i.

R2i asserts genomic integrity after long-term cultivation: Displaying a stable karyotype during repetitive passages of embryonic stem (ES) cells is one of the serious issues in developing of a culture medium. Although the use of small molecule inhibitors in defined medium has advanced the generation of pluripotent stem cells through rodentia, there is a concern that these chemical perturbations may threaten genomic integrity of cells. Specially, the application of CHIR in 2i compound medium gives rise to an apprehension of chromosomal abnormalities heightening in ES cells owing to the fact that GSK3 inhibition by related small molecules or RNA interference endangers chromosomal alignment at mitosis. To evaluate the effects of R2i on ES cell culture and for a comparison with 2i culture medium condition, the karyotype of 10 selected virtually normal ES cell lines after long-term passaging (2:20) in R2i and 2i medium was assessed. The data shows that while ES cells in 2i may undergone many alternations, R2i could retain a normal diploid karyotype until high passage number. Therefore, R2i do not distribute chromosomal integrity and could be assumed as a reliable substituting of 2i in cultivation of naive mouse ES cells.

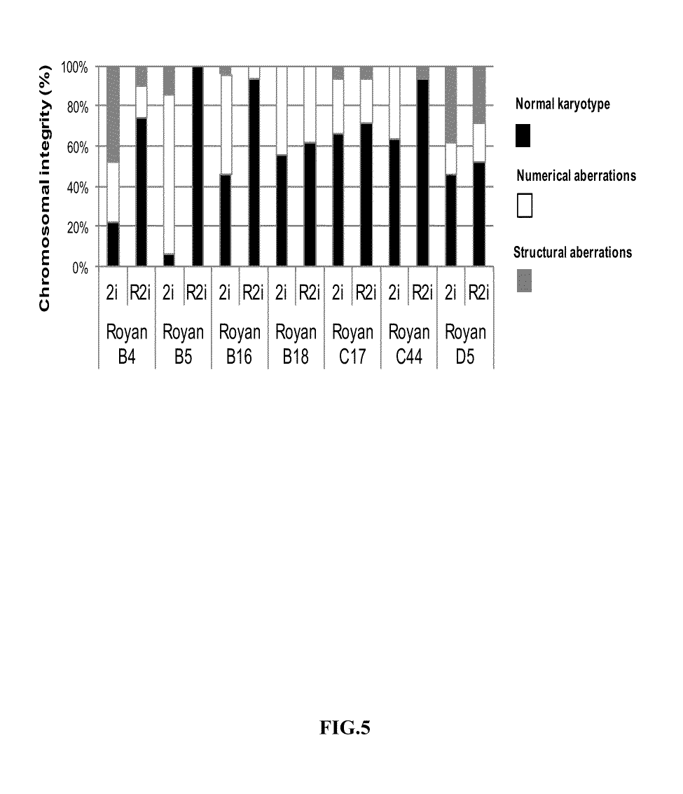

FIG. 5 illustrates a bar chart indicating the role of R2i in genomic integrity after long term cultivation, according to the embodiments herein. FIG. 5 illustrates that the R2i supplemented medium supports the maintenance of normal karyotype more efficiently than 2i supplemented medium, after long term passaging (at least 20 passages) of different embryonic stem cell lines in medium supplemented with 2i and R2i.

TGF-β Inhibition sustains the pluripotency of mouse Embryonic Stem (ES) Cells: The affirmative role of TGF-β signaling suppression by R2i compound is at odds with the most previous reports about the function of this pathway in mouse ES cells. Although most studies indicate the necessity of TGF-β signaling in undifferentiated mouse embryonic stem (ES) cells, the present study proclaims the astonishing effects of inhibition of this pathway on pluripotency. For more evaluation of TGF-β inhibition effects, the growth rate of R2i supplemented medium cultured cells was assessed. Two R2i supplemented media cell lines, Royan D4 and Royan B20, that coincidentally were passaged 7 times in serum, 2i±LIF and R2i±LIF, were measured for the growth ratios by determining cell numbers on 24, 48 and 72 hours after cell seeding. The results indicated that the proliferation rate were not affected significantly in R2i with regard to serum or 2i, although it appears that leukemia inhibitory factor (LIF) is indispensable for optimal growth rate in Royan B20, both in 2i and R2i supplemented medium.

The other inhibitor for TGF-β signaling pathway was applied in combination with programmed death (PD) inhibitor for a better comprehending of TGF-β inhibition in pluripotency of mouse ES cells were applied. The Royan B20 which passed more than 7 passages in PD+TGF-β inhibitors including A83-01 and ALK5 inhibitor (ALK5i)+LIF, exhibited the main signature of ES cells resemblance to inhibition of TGF-β by small molecule inhibitor (SB) in R2i compound. The qRT-PCR analysis confirmed that all three TGF-β inhibitors in combination with PD manifested the same attitude regarding to the expression of pluripotency and lineage specified marker genes.

To assure the impressive roles of TGF-β inhibition in pluripotency, the inhibition of this pathway was evaluated. Extensive cell death was observed if TGF-β small molecule inhibitors were used alone in N2B27 culture medium. On the contrary, whenever leukemia inhibitory factor (LIF) was added to these inhibitors, the cells could be cultivated readily without the observation of mortality or leaving pluripotency. Although the proliferation rate of cells were markedly declined. For assessing the effect of TGF-β inhibition downstream of receptor activation, the siRNAs were applied against Smad2 and Smad3 mRNAs. The Royan B20 cell lines (which are passaged 3 times in N2B27+SB431542+LIF) were seeded on gelatin coated 6 well plates in 1 ml of N2B27 medium containing only LIF. Three different siRNAs were used for each Smad2 and Smad3. The medium was changed after elapse of 24 hour and 48 hour since the transfection of the cells. The cells were collected and subjected to mRNA isolation, through standard protocol. The isolated mRNA is subjected to qRT-PCR analysis. Table 5 below illustrates the sequences of siRNA against Smad2 and Smad3:

TABLE 5
Table S10. Sequences of siRNAs against Smad2 and
Smad3

| Name | Sequence |
|---|---|
| Smad2,A | S5′,CCUCCGUCGUAGUAUUCAU UU |
| | AS3′,UU GGAGGCAGCAUCAUAAGUA |
| Smad2,B | S5′,GCCUAAGUGAUAGUGCAAU UU |
| | AS3′,UU CGGAUUCACUAUCACGUUA |
| Smad2,C | S5′,GACGGUUAGAUGAGCUUGA UU |
| | AS3′,UU CUGCCAAUCUACUCGAACU |
| Smad3,A | S5′,GUGAGCGCUUUACAUUAGA UU |
| | AS3′,UU CACUCGCGAAAUGUAAUCU |
| Smad3,B | S5′,GUCAGCGUAUAGGUGAUGU UU |
| | AS3′,UU CAGUCGCAUAUCCACUACA |
| Smad3,C | S5′,GUGUGAGUCUCCGUUGAUA UU |
| | AS3′,UU CACACUCAGAGGCAACUAU |

The qRT-PCR analysis affirmed that different TGF-β small molecule inhibitors or Smad2 and Smad3 siRNAs, all plus LIF, maintain the pluripotency in mouse ES cell culture after several passaging in N2B27 defined medium. However, TGF-β inhibitors+LIF could not be assumed as an optimum culture medium for mouse pluripotent stem cells since this condition does not lead to ES cell line derivation and is also unable to form undifferentiated colonies from isolated single ES cells. The data firmly indicate that inhibition of TGF-β signaling supports the pluripotency of mouse ES cells.

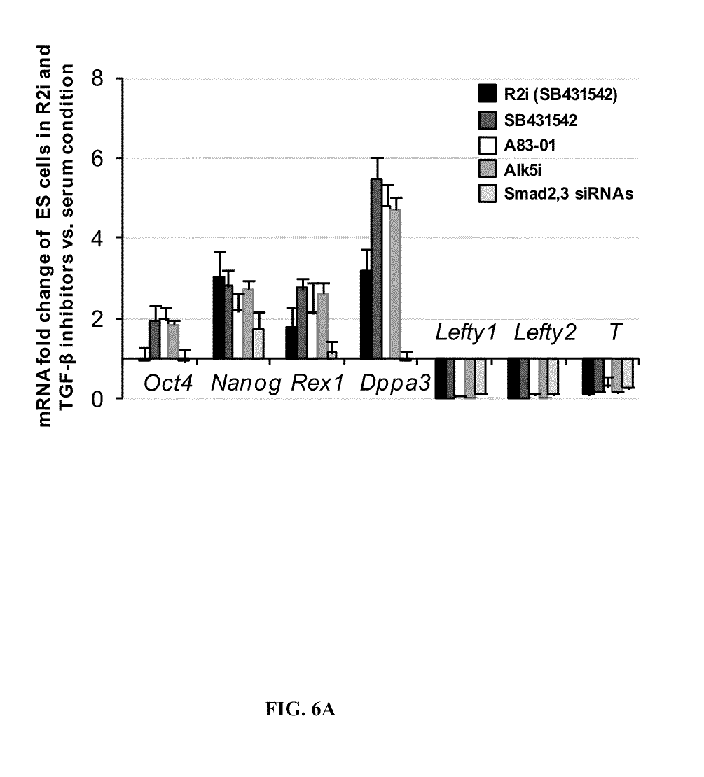

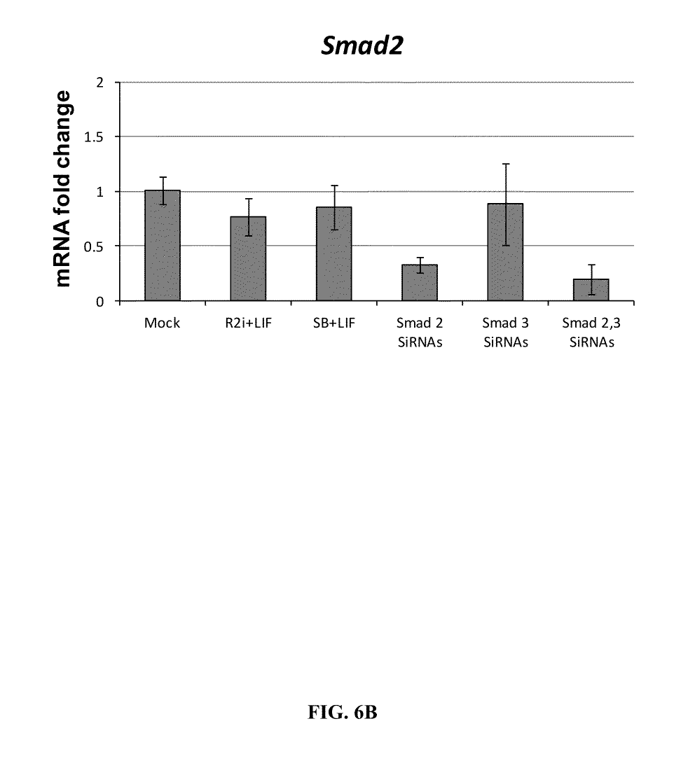

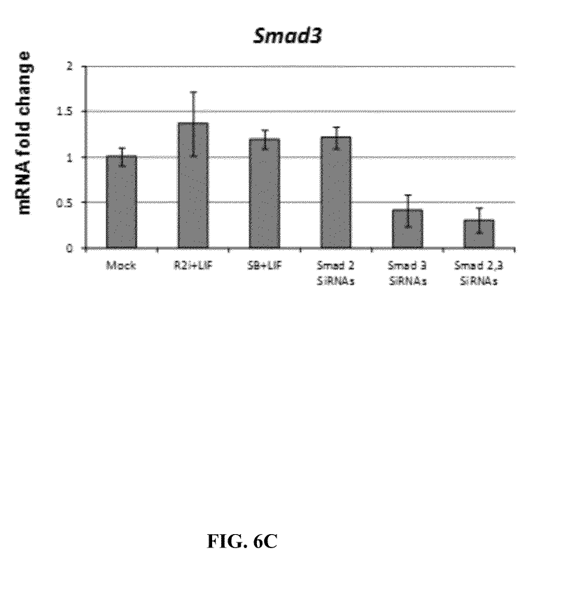

FIG. 6A illustrates a bar chart indicating the results for TGF β inhibition sustaining the pluripotency of mouse embryonic stem cells, according to an embodiment herein. FIG. 6A illustrates the mRNA fold change of embryonic stem cells in R2i and TGF-β inhibitors versus serum in the medium. FIG. 6A illustrates the qRT-PCR results for pluripotency and differenatiation specific genes in Royan B20 after cultivation for seven passages in different TGF-β inhibitors (SB431542, A83-01, and ALK5i), Smad2, Smad3 and the serum is considered as control. All the media are supplemented with leukemia inhibitory factor (LIF). The relative expression levels of the genes are normalized to the housekeeping gene Gapdh. The mRNA's considered for expression analysis are Oct4, Nanog, Rex1, Dppa3, Lefty 1, Lefty 2 and T. The FIG. 6A illustrates that the mRNA fold change in the embryonic stem cells is minimal in the presence of R2i.

FIG. 6B and FIG. 6C illustrate a bar chart indicating mRNA fold change in the embryonic stem cells in the media of the Smad2 and Smad3 which are knocked down by the use of siRNA in embryonic stem cells. FIG. 6B-6C illustrates that the knockdown of the Smad2 and Smad3 has a similar effects as the R2i inhibitors in the expression of pluripotency markers.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A method for deriving and long term establishment of ground state pluripotent embryonic stem cells comprises:
isolating mouse embryonic stem cells; and
culturing the mouse embryonic stem cells in a culture media comprising the small molecule inhibitors of signal regulated pathways, for a long term maintenance of the mouse embryonic stem cell.

2. The method according to claim 1, wherein the step of isolating and long term maintenance of mouse embryonic stem cell comprises:
maintaining a plurality of mouse strains on a 12 hour light/dark regimen, wherein the plurality of mouse strains are selected from a group consisting of BALB/c, C57BL/6, DBA/2, and F1 generation of C57BL/6 (Oct−EGFP)×CD−1;
recovering a mouse embryo by flushing uteri or oviduct of a mouse for blastocysts or cleavage embryos at E3.5 stage;
obtaining blastocyst at E3.5 stage from the plurality of mouse strains;
deriving the mouse embryonic stem cells from 3.5 day blastocyst in a media comprising R2i molecule;
plating a zona-free E.3.5 day blastocyst on a gelatine coated plate comprising a predefined medium with the R2i and a leukemia inhibitory factor (LIF), and wherein the predefined medium is selected from a group consisting of KSOM medium, N2B27 medium and serum;
accomplishing immunology for removing tropectoderm and establishing embryonic stem cell lines;
disaggregating a cell mass by a 0.05% trypsin 5-7 days after the blastocyst or an inner cell mass plating to obtain disassociated cells;
centrifuging the disassociated cells to remove the serum and harvest the cells;and
transferring the harvested cells in bacterial dishes comprising N2B27 medium, the R2i molecule, and the LIF for cultivation as suspension.

3. The method according to claim 2, wherein the harvested cells are transferred on gelatine coated plates comprising the N2B27 medium, the R2i molecule and the LIF for cultivation as adherent.

4. The method according to claim 1, wherein the small molecule inhibitor are R2i molecular combination and 2i molecular combination, wherein the 2i molecular combination comprises a PD0325901 and a CHIR99021, and wherein the R2i molecular combination comprises a SB431542 and a PD0325901.

5. The method according to claim 1, wherein the small molecule inhibitor inhibits metabolic pathways, wherein the metabolic pathways are an extracellular signal regulated kinase (ERK) pathways and a transforming growth factor β (TGFβ) signalling pathway.

6. The method according to claim 2, wherein a 100 ml of the N2B27 medium comprises:
45 ml of Dulbeccos modified eagle medium-nutrient mixture F-12;
45 ml of neuro basal medium;
1 ml of nitrogen supplement (1 vol %);
2 ml of B27 supplement (1 vol %);
1 ml of L-glutamine (2 mM);
1 ml of non-essential amino acid (1 vol %);
1 ml of penicillin/streptomycin;
B mercaptoethanol (0.1 mM);
5 mg/mL of Bovine serum albumin (BSA);
1000 μ/ml of leukemia inhibitory factor (LIF); and
the R2i molecular combination, wherein R2i molecular combination comprises 1 μM PD 0325901 and 10 μM SB431542.

7. The method according to claim 1, wherein the derived embryonic stem cells are cultured separately on a mouse embryonic fibroblast (MEF) coated 96 well plate in a desired medium, wherein the desired medium is selected from a group consisting of KSOM, mouse embryonic stem cells with serum medium and N2B27 medium supplemented with the 2i molecule combination or the R2i molecule combination.

8. The method according to claim 1, wherein the embryonic stem cells are routinely passaged in every 2-3 days.

9. The method according to claim 1, wherein the mouse embryonic stem cells derived from the single blastomeres of 2 cell, 4 cell and 8 cell embryos from the plurality of mouse strains in the R2i molecular combination supplemented media is two times more than that of the 2i molecular combination supplemented media.

10. The method according to claim 1, wherein the mouse embryonic stem cells generated in the culture media comprising the R2i molecular combination are homogeneous in terms of expression of pluripotency markers, and wherein the pluripotency markers comprise a Nanong and a Stella.

11. The method according to claim 1, wherein a chromosomal stability of the embryonic stem cells in culture media comprising of the R2i molecular combination is higher than the that in the media comprising of the 2i molecular combination.

12. The method according to claim 1, wherein an inhibition of transforming growth factor β (TGFβ) receptors in combination with the leukemia inhibitory factor (LIF) supports the ground state of the embryonic stem cells and a self renewal of the embryonic stem cells.

13. The method according to claim 2, wherein gelatine coated plate comprises only the predefined medium with the R2i, and wherein the predefined medium is selected from a group consisting of KSOM medium, N2B27 medium and serum.
